SEQUENCE LISTING (16 sequences)

SEQ ID NO: 1 - DNA, 28 bp, Artificial Sequence
  Description of Artificial Sequence Synthetic primer
gctgcaggagagtggcgcctccgctcat

SEQ ID NO: 2 - DNA, 17 bp, Artificial Sequence
  Description of Artificial Sequence Synthetic primer
acggagctggggcggcc

SEQ ID NO: 3 - DNA, 21 bp, Artificial Sequence
  Description of Artificial Sequence Synthetic primer
gagagtggcgcctccgctcat

SEQ ID NO: 4 - DNA, 17 bp, Artificial Sequence
  Description of Artificial Sequence Synthetic primer
acggagctggggcggcc

SEQ ID NO: 5 - DNA, 29 bp, Artificial Sequence
  Description of Artificial Sequence Synthetic primer
aggatccccgcagaggatttcgtgtacca

SEQ ID NO: 6 - DNA, 25 bp, Artificial Sequence
  Description of Artificial Sequence Synthetic primer
tccccgcagaggatttcgtgtacca

SEQ ID NO: 7 - DNA, 25 bp, Artificial Sequence
  Description of Artificial Sequence Synthetic primer
cggatccggcccaaagccctcactc

SEQ ID NO: 8 - DNA, 20 bp, Artificial Sequence
  Description of Artificial Sequence Synthetic primer
ccggcccaaagccctcactc

SEQ ID NO: 9 - DNA, 20 bp, Artificial Sequence
  Description of Artificial Sequence Synthetic primer
ccggcccaaagccctcactc

SEQ ID NO: 10 - DNA, 20 bp, Artificial Sequence
  Description of Artificial Sequence Synthetic primer
agctccgtcaccgcccggaa

SEQ ID NO: 11 - DNA, 26 bp, Artificial Sequence
  Description of Artificial Sequence Synthetic primer
cggtacacctccacgtcgctgtcsaa

SEQ ID NO: 12 - DNA, 26 bp, Artificial Sequence
  Description of Artificial Sequence Synthetic primer
cggtacacctccacgtcgctgtcsaa

SEQ ID NO: 13 - DNA, 25 bp, Artificial Sequence
  Description of Artificial Sequence Synthetic primer
tccccgcagaggatttcgtgtacca

SEQ ID NO: 14 - DNA, 26 bp, Artificial Sequence
  Description of Artificial Sequence Synthetic primer
cggtacacctccacgtcgctgtcsaa

SEQ ID NO: 15 - DNA, 16 bp, Artificial Sequence
  Description of Artificial Sequence Synthetic probe
cgtgcgttatgtgacc

SEQ ID NO: 16 - DNA, 15 bp, Artificial Sequence
  Description of Artificial Sequence Synthetic probe
cgtgcgtcttgtgac

(19) United States
(12) Patent Application Publication
(10) Pub. No.: US 2015/0203914 A1
(43) Pub. Date: Jul. 23, 2015

(54) METHOD OF DETERMINING THE FRACTION OF FETAL DNA IN MATERNAL BLOOD USING HLA MARKERS

(71) Applicant: Roche Molecular Systems, Pleasanton, CA (US)

(72) Inventors: Henry Erlich, Oakland, CA (US); Bryan Hoglund, Pleasanton, CA (US); Cherie Holcomb, Oakland, CA (US); Priscilla Moonsamy, Pleasanton, CA (US); Nick Newton, Oakland, CA (US); Melinda Rastrou, Pleasanton, CA (US); Nancy Schoenbrunner, Moraga, CA (US); Alison Tsan, Danville, CA (US)

(73) Assignee: Roche Molecular Systems, Pleasanton, CA (US)

(21) Appl. No.: 14/273,763

(22) Filed: May 9, 2014

Related U.S. Application Data
(60) Provisional application No. 61/821,620, filed on May 9, 2013.
(60) Provisional application No. 61/861,316, filed on Aug. 1, 2013.

(51) Int. Cl. C12Q 1/68

(52) U.S. Cl. CPC C12Q 1/6881; C12Q 2600/16; C12Q 2600/158; C12Q 2600/112

(57) ABSTRACT

The invention comprises a method of determining the fraction of fetal DNA in maternal blood or plasma using HLA locus wherein the maternal and fetal HLA alleles are detected and quantified by clonal or digital methods.

### SEQUENCE LISTING

The instant application contains a Sequence Listing which has been submitted electronically in ASCII format and is hereby incorporated by reference in its entirety. Said ASCII copy, created on Apr. 25, 2014, is named 31556-USI_SL.txt and is 4,092 bytes in size.

The discovery of cell-free fetal DNA in maternal blood in 1997 has launched a new field of non-invasive prenatal diagnosis and screening. The uses of circulating fetal DNA include Rh group genotyping as well as screening for aneuploidy and monogenic diseases, reviewed in Jiang, P., et al. (2012) FetalQuant: deducing fractional fetal DNA concentration from massively parallel sequencing of DNA in maternal plasma. Bioinformatics 28:2883. Such diagnostic applications require the knowledge of the fraction of fetal DNA within the total DNA present in maternal circulation (also known as the “fetal fraction”). For some applications, that would be merely a quality control issue. For example, some of the current commercial non-invasive prenatal diagnostic (NIPD) tests offered by CLIA labs indicate that if <4% of the DNA in maternal plasma is fetal, the test results would be inconclusive and so the test is not run. For other applications, such as detection of aneuploidy and the copy number of recessive alleles, precise determination of the fetal fraction is essential.

Current approaches to quantifying the fetal DNA in maternal plasma involve detection of Y chromosome loci (e.g. SRY or DYS14, U.S. Pat. No. 6,258,540); detection of fetal epigenetic markers (e.g. methylation pattern of genes maspin, RASSF1A and SERPINB2, reviewed in Hahn, S., et al., (2011) Cell-free nucleic acids as potential markers for preeclampsia, Placenta, 32:sl7); or targeting a large number of chromosomal loci in the hope of finding informative single nucleotide polymorphisms (SNPs) that can distinguish maternal DNA from fetal DNA (U.S. application Ser. No. 12/644,388). The obvious disadvantage of the Y-chromosome-based approach is its inapplicability to female fetuses. The epigenetic approach is notorious for lack of reproducibility. The methods using multiple SNPs (e.g. liang, et al., supra) require complex mathematical analysis in search of informative SNPs throughout the genome. A more predictable and precise means of determining fetal fraction is desirable.

### SUMMARY OF THE INVENTION

The present invention is a method of determining a fraction of fetal nucleic acid in a sample comprising: quantitatively detecting at least one non-maternal HLA allele in the sample; quantitatively detecting in the sample at least one maternal HLA allele at the same locus; using the quantities detected above, determining fraction of fetal nucleic acid in the sample. In some embodiments, the determining step comprises calculating 2 times the ratio of the quantity of the non-maternal HLA allele to the sum of quantities of all HLA alleles determined in steps. In some embodiments, the at least one non-maternal and maternal HLA allele is selected from HLA-A, HLA-B, HLA-C, DRB1, DRB3, DRB4, DRB5, DQA1, DQB1, DPA1, and DPB1 allele, or HLA-A, exons 2 and 3; HLA-B, exons 2 and 3; HLA-C, exons 2 and 3; DQA1, exon 2; DQB1, exons 2 and 3; DPA1, exon 2; DPB1, exon 2; DRB1, exon 2; DRB3, exon 2; DRB4, exon 2; and DRB5, exon 2 or an intron sequence from said HLA genes or a combination of exon and intron sequences from said genes. In some embodiments, the HLA alleles are quantitatively detected by a method comprising clonal sequencing and optionally including a step of clonal amplification. In some embodiments, the method comprises a target enrichment step prior to sequencing, e.g., by at least one round of genomic DNA amplification or by target capture.

In some embodiments, the HLA alleles are quantitatively detected by a method comprising: amplification with a forward primer and reverse primer to obtain HLA amplicons; performing clonal sequencing to determine the sequence of the HLA amplicons: identifying at least one maternal HLA allele and at least one non-maternal HLA allele at the same locus; comparing the number of maternal and non-maternal HLA sequence clones thereby determining fraction of fetal nucleic acid in the sample. In some embodiments, the identifying step comprises computational steps of: comparing the sequences at the HLA locus to an HLA sequence database; sorting the sequences into multiple bins corresponding to known HLA alleles; identifying one or two majority sequences as maternal alleles; identifying one or two most represented minority sequences as non-maternal alleles. In some embodiments, determining the sequence of the HLA amplicons comprises sequencing by synthesis. In some embodiments, the non-maternal HLA allele and the maternal HLA allele at the same locus are detected at two, three or more loci, e.g., DPB1, DQB1 and DRB1.

In another embodiment, the HLA alleles are quantitatively detected by a method comprising: partitioning the sample into a plurality of reaction volumes, each comprising between zero and approximately five copies of the target HLA allele; assaying each reaction volume for the presence of the target HLA allele; comparing the number of reaction volumes containing the non-maternal HLA allele to the number of reaction volumes containing the maternal HLA allele at the same locus, thereby determining fraction of the fetal nucleic acid in the sample. In variations of this embodiment, the assaying comprises amplification by digital PCR.

In some embodiments, the method further comprises independently obtaining genotype information for one or both parents at the at least one HLA locus.

In yet another embodiment, the invention is a method of detecting a chromosomal abnormality in a fetus comprising: obtaining a blood sample from the mother carrying the fetus; determining fraction of fetal nucleic acid in the sample by a method comprising quantitatively detecting at least one non-maternal HLA allele in the sample; quantitatively detecting in the sample at least one maternal HLA allele at the same locus; comparing the quantities of the maternal and non-maternal HLA alleles thereby determining concentration of fetal nucleic acid in the sample; quantitatively detecting in the sample a locus from at least one chromosome suspected of an abnormality; determining whether the chromosomal locus detected in step (c) is present in an abnormal amount relative to the concentration of fetal DNA determined in step (b) thereby detecting the chromosomal abnormality. In variations of this embodiment, the at least one non-maternal HLA allele is selected from HLA-A, HLA-B, HLA-C, DRB1, DRB3, DRB4, DRB5, DQA1, DQB1, DPA1, and DPB1 allele, including HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2 or a combination of exon and intron sequences from said genes. In variations of this embodiment, the non-maternal HLA allele and the maternal HLA allele at the same locus are detected at two, three or more loci, e.g., from genes DPB1, DQB1 and DRB1.

In yet another embodiment, the invention is a method of determining whether a pregnant patient has or is likely to develop preeclampsia by determining whether fetal fraction in the patient's blood exceeds a threshold level, wherein the fetal fraction is determined by a method comprising obtaining a blood sample from the patient; quantitatively detecting at least one non-maternal HLA allele in the sample; quantitatively detecting in the sample at least one maternal HLA allele at the same locus; comparing the quantities of the maternal and non-maternal HLA alleles thereby determining fetal fraction in the sample. In variations of this embodiment, the at least one non-maternal HLA allele is selected from HLA-A, HLA-B, HLA-C, DRB1, DRB3, DRB4, DRB5, DQA1, DQB1, DPA1, and DPB1 allele, e.g., HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2 or a combination of exon and intron sequences from said genes. In variations of this embodiment, the non-maternal HLA allele and the maternal HLA allele at the same locus are detected at two, three or more loci, e.g., DPB1, DQB1 and DRB1. In yet another variation of this embodiment, the invention is a method of monitoring a pregnant patient for development of preclampsia by periodically determining fetal fraction in the patient's blood by the method described above and if an increase in the fetal fraction is detected, diagnosing the patient as having or likely to develop preeclampsia.

In yet another embodiment, the invention is a method of determining whether a pregnant patient has or is likely to develop preeclampsia by determining whether concentration of fetal nucleic acid in maternal blood exceeds a threshold level, wherein the concentration of fetal nucleic acid is determined by a method comprising obtaining a volume blood sample from the patient; quantitatively detecting at least one non-maternal HLA allele in the volume of the sample thereby determining the concentration of fetal nucleic acid. In variations of this embodiment, the at least one non-maternal HLA allele is selected from HLA-A, HLA-B, HLA-C, DRB1, DRB3, DRB4, DRB5, DQA1, DQB1, DPA1, and DPB1 allele, e.g., HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2 or a combination of exon and intron sequences from said genes. In further variations of this embodiment, the non-maternal HLA allele and the maternal HLA allele at the same locus are detected at two, three or more loci, e.g., DPB1, DQB1 and DRB1.

In yet another embodiment, the invention is a method of detecting a presence or a homozygous state of an allele in a fetus wherein the allele is associated with a disease state, the method comprising: obtaining a blood sample from the mother carrying the fetus; determining a fraction of a non-maternal HLA allele in the sample by a method comprising quantitatively detecting at least one non-maternal HLA allele in the sample; quantitatively detecting in the sample at least one maternal HLA allele at the same locus; comparing the quantities of the maternal and non-maternal HLA alleles thereby determining the fraction of the non-maternal HLA allele in the sample; quantitatively detecting in the sample the allele associated with the disease state; comparing the quantity of the allele associated with the disease state with the fraction of the non-maternal HLA allele thereby determining whether said allele associated with disease state is present in a single copy, two copies or is absent from the fetus. In variations of this embodiment, the at least one non-maternal HLA allele is selected from HLA-A, HLA-B, HLA-C, DRB1, DRB3, DRB4, DRB5, DQA1, DQB1, DPA1, and DPB1 allele, e.g., HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2 or a combination of exon and intron sequences from said genes. In further variations of this embodiment, the non-maternal HLA allele and the maternal HLA allele at the same locus are detected at two, three or more loci, e.g., DPB1, DQB1 and DRB1.

### DETAILED DESCRIPTION OF THE INVENTION

### Definitions

The term “allele” refers to a sequence variant of a gene. One or more genetic differences can constitute an allele. For HLA alleles, typically, multiple genetic differences constitute an allele (i.e., most alleles differ from one another by more than one base). As used herein, a maternal allele is one of the two alleles present in the mother. A non-maternal allele is the allele present in the fetus but not present in the mother. The non-maternal allele can be a paternal allele present in the fetus. The non-maternal allele can also be a new allele resulting from homologous recombination or gene conversion during meiosis or a germline mutation in either parent and passed down to the fetus. The non-maternal allele can also be derived from a donor, e.g. an egg donor, contributing genetic material to the fetus.

The term “clonal” in the context of “clonal analysis” refers to separately analyzing an aggregate or population of molecules all derived from a single molecule. For example, “clonal sequencing” refers to individually sequencing each amplicon that was derived from the same molecule (target amplicon).

The term “deep sequencing” refers to a sequencing method wherein the target sequence is read multiple times in the single test. A single deep sequencing run is composed of a multitude of sequencing reactions run on the same target sequence and each, generating independent sequence readout.

The term “digital” in the context of “digital analysis” or “digital dilution” refers to the analysis of each of a plurality of individual molecules present in a sample. Digital dilution refers to distribution of the sample into a plurality of reaction volumes where, on average, one or fewer molecules are present in each reaction volume. In some instances, digital dilution enables a digital readout, e.g. obtaining a yes/no result from each individual molecule and tabulating the digital results obtained from a population of molecules by counting the number of clonal sequences.

The term “digital droplet PCR” or “ddPCR” refers to PCR performed in a plurality of reaction volumes (“droplets”) resulting from digital dilution of a sample.

The term “fetal fraction” refers to the proportion of fetal nucleic acid among the total nucleic acid. For example, fetal fraction may represent the proportion of fetal DNA in the total DNA present in (or isolated from) maternal plasma. It is understood that for a heterozygous fetus, the fraction of one of the fetal alleles will represent one half of the fetal fraction.

The terms “maternal” and “mother” refer to the woman carrying the fetus. The method of the invention is applicable to both genetic mothers of the fetus as well as women carrying a fetus not related to them genetically, e.g. a fetus originating from a donor egg or otherwise carrying donor's genetic material.

The term “polymorphism” refers to the condition in which two or more variants of a genomic sequence, or the encoded amino acid sequence, can be found in a population. A “single nucleotide polymorphism,” (SNP) is a polymorphism where the variation in the sequence consists of a single polymorphic nucleotide position in the genomic sequence.

The term “genotype” refers to a combination of one or more alleles of one or more genes contained in an individual or a sample derived from the individual.

The term “haplotype” refers to a combination of one or more alleles of one or more genes present on the same chromosome of an individual.

The term “determining the genotype of an HLA gene” refers to determining the selected combination of HLA alleles in a subject. For example, in the present invention, “determining the genotype of an HLA-A gene” refers to identifying at least one of the polymorphic residues (allelic determinants) present in one or more of the exons, e.g., exons 2, 3 and 4 of the HLA-A gene. In a similar fashion, genotypes of the genes HLA-B, HLA-C, DRB1, DRB3, DRB4, DRB5, DPB1, DPA1, DQA1 and DQB1 can be determined.

The term “target region” refers to a region of a nucleic acid sequence that is to be analyzed.

The term “nucleic acid” refers to polymers of nucleotides (e.g., ribonucleotides or deoxyribo-nucleotides) both natural and non-natural. The term is not limited by length (e.g., number of monomers) of the polymer. A nucleic acid may be single-stranded or double-stranded and will generally contain 5′-3′ phosphodiester bonds, although in some cases, nucleotide analogs may have other linkages. Nucleic acids may include naturally occurring bases (adenosine, guanidine, cytosine, uracil and thymidine) as well as non-natural bases. The term “non-natural nucleotide” or “modified nucleotide” refers to a nucleotide that contains a modified nitrogenous base, sugar or phosphate group, or that incorporates a non-natural moiety in its structure. Examples of non-natural nucleotides include dideoxynudeotides, biotinylated, aminated, deaminated, alkylated, benzylated and fluorophor-labeled nucleotides.

The term “primer” refers to a short nucleic acid (an oligonucleotide) that acts as a point of initiation of DNA synthesis by a nucleic acid polymerase under suitable conditions that typically include an appropriate buffer, the presence of nucleic acid precursors and one or more optional cofactors and a suitable temperature. A primer typically includes at least one target-hybridized region that is at least substantially complementary to the target sequence. This region is typically about 15 to about 40 nucleotides in length.

The term “adapter region” or “adapter” of a primer refers to the region of a primer typically located to the 5′ of the target-hybridizing region. Typically the adapter serves a function in a subsequent analysis step. For example, the adapter may hybridize to an oligonucleotide conjugated to a microparticle or other solid surface used for amplification, e.g., emulsion PCR. The adapter can also serve as a binding site for a primer used in subsequent steps, e.g., a sequencing primer. The adapter region is typically from 15 to 30 nucleotides in length.

The terms “individual identifier tag,” “identification tag,” “multiplex identification tag” or “MID” are used interchangeably herein to refer to a region of a primer that serves as a marker of the DNA obtained from a particular sample.

The term “amplification conditions” refers to conditions in a nucleic acid amplification reaction (e.g., PCR amplification) that allow for hybridization and template-dependent extension of the primers. The term “amplicon” refers to a nucleic acid molecule that contains all or a fragment of the target nucleic acid sequence and that is formed as the product of in vitro amplification by any suitable amplification method. Various PCR conditions are described in PCR Strategies (M. A. Innis, D. H. Gelfand, and J. J. Sninsky eds., 1995, Academic Press, San Diego, Calif.) at Chapter 14; PCR Protocols: A Guide to Methods and Applications (M. A. Innis, D. H. Gelfand, J. J. Sninsky, and T. J. White eds., Academic Press, NY, 1990)

The term “sample” refers to any composition containing or presumed to contain nucleic acid from an individual. In the context of the present invention, the sample wherein the fetal fraction is determined is maternal blood and fractions derived therefrom, e.g. blood plasma. However, for certain aspects of the invention, e.g. for determination of parental genotypes, any other type of body sample may be used, including without limitation, skin, plasma, serum, whole blood and blood components, saliva, urine, tears, seminal fluid, vaginal fluids and other fluids and tissues, including paraffin embedded tissues. Samples also may include constituents and components of in vitro cultures of cells obtained from an individual.

The term “valid read” in connection with nucleic acid sequencing refers to a sequence read successfully assigned (with or without error corrections) to a particular genome sequence. In reference to HLA alleles, a valid read is a sequence read successfully assigned (with or without error corrections) to one of the HLA alleles expected to be present in the sample. A read that may not be assigned to any alleles expected to be present in the sample or to any of the sequences in the IMGT HLA sequence database is an invalid read.

The invention provides methods of estimating the fetal fraction using unique properties of the Human Leukocyte Antigen (HLA) locus. The genes of the HLA region (HLA genes) are the most polymorphic in the human genome. The HLA region spans approximately 3.5 million base pairs on the short arm of chromosome 6. The major regions are the Class I and Class II regions. The Class I genes are HLA-A, HLA-B, and HLA-C and the major Class II genes are HLA-DP, HLA-DQ and HLA-DR. Polymorphisms that are expressed at the protein level are reflected in the amino acid sequence of the HLA antigen and therefore are of great interest for tissue typing for transplantation. These polymorphisms are localized primarily in exon 2 for the Class II genes and exons 2 and 3 for the Class I genes. However, for the purposes of fetal fraction determination, all polymorphisms, including the silent changes (nucleotide changes not resulting in an amino acid change) as well as changes in introns and other non-coding regions of the HLA genes are useful.

The choice of HLA locus as the target non-maternal sequence to be detected is far superior to the existing targets. Therefore the present invention is a substantial improvement on existing methods of determining fetal fraction. For example, the methods that detect Y-chromosome sequences exclude female fetuses. By contrast, HLA genes are located on an autosome (chromosome 6) and thus can be detected in both genders. In addition, since both the father and mother, unlike Y chromosome markers, have HLA genes, the sequence reads in the numerator are from the same amplicon as the denominator in the fetal fraction calculations. The widely used SNP-based method (e.g. U.S. application Ser. No. 12/644,388) aims to detect single-nucleotide differences (SNPs) between maternal and fetal sequences. Yet most amplification and sequencing technologies are error-prone such that many perceived single-nucleotide changes are artifacts and not true SNPs. By contrast, HLA alleles differ from one another by multiple nucleotides. Thus a method comprising detection of alleles within the HLA locus is less vulnerable to error compared to non-HLA loci. The currently available HLA genotyping methods using, e.g., clonal sequencing, enable setting the phase of multiple linked polymorphisms within an exon and make possible the unambiguous determination of the sequence of each HLA allele. This feature adds an additional degree of accuracy in distinguishing fetal DNA from maternal DNA and accurately quantifying the fetal fraction.

The present invention is a method of quantifying the fraction or amount of variant (non-maternal) HLA sequence among the cell-free DNA present in maternal blood or plasma. The method further comprises obtaining an estimate of the proportion of fetal DNA (fetal fraction) by determining the proportion of the non-maternal HLA sequence compared to the maternal HLA sequence or total amount of HLA sequence. In one embodiment, the method further comprises using the estimated fetal fraction as a reference for determining fetal aneuploidy. In other embodiments, the estimated fetal fractions used as a threshold for rejecting (i.e., excluding from diagnostic procedures) samples with insufficient amounts of fetal DNA. In yet another embodiment, the method comprises using the estimated fetal fraction or amount or concentration of fetal DNA to diagnose the likelihood of preeclampsia. In variations of this embodiment, the method further comprises monitoring the fetal fraction or amount or concentration of fetal DNA and if an increase has been detected, identifying the patient as having or likely to develop preeclampsia. In yet another embodiment, the method comprises using the estimated fetal fraction to determine the presence or homozygous state of an allele in the fetus. In variations of this embodiment, the method comprises detection of a homozygous state (e.g., the state associated with mortality and morbidity) of a recessive allele in the fetus. In other variations of this embodiment, the method comprises detecting a single copy of an allele (e.g., the carrier state for recessive alleles or the state associated with mortality and morbidity for autosomal dominant or haploinsufficient alleles) in the fetus. An accurate and precise estimate of the fetal fraction determined with HLA markers is critical in interpreting results obtained for the mutant disease-associated allele. Any polymorphic HLA gene or locus may be used with the method of the present invention. In some embodiments, the HLA gene is selected from HLA-A, HLA-B, HLA-C, DRB1, DRB3, DRB4, DRB5, DQA1, DQB1, DPA1, and DPB1. In some embodiments, specific exons or portions of exons of HLA genes are targeted, e.g., HLA-A, exons 2 and 3; HLA-B, exons 2 and 3; HLA-C, exon, 2 and 3; DQA1: exon2; DQB1: exons 2 and 3; DPA1: exon 2; DPB1: exon 2; DRB1: exon 2, DRB3: exon 2; DRB4: exon 2; and DRB5: exon 2. In other embodiments, the polymorphic HLA gene sequence comprises introns sequences or a combination of exon and intron sequences.

In some embodiments, multiple HLA genes or loci are analyzed in the same reaction in the form of a gene panel. In one embodiment, the panel is formed of gene sequences that are not closely linked and are not in linkage disequilibrium. This approach is especially advantageous when parental HLA genotypes are not known: the use of several unlinked loci assures that at least some loci will be informative (i.e., polymorphic between the mother and the fetus with an allele present in the fetus that is absent in the mother). For example, in a variation of this embodiment, sequences from genes DPB1 and DQB1 or DPB1 and DRB1 are analyzed simultaneously. In another embodiment, the panel is formed of closely linked gene sequences that are in strong linkage disequilibrium. This approach assures that experimental errors such as a sequencing error in the maternal sequence that creates a sequence corresponding to a known HLA allele different from the maternal allele are recognized and discarded as “noise” rather than “signal.” For example, if sequence reads from the DQA1 locus differ from the maternal allele by one base, these could, in principle, reflect the unknown paternal allele present in the fetal DNA or, in contrast, a sequencing error in the maternal allele. If these DQA1 non-maternal sequences are, in fact, derived from the fetal DNA, then one would expect, based on known linkage disequilibrium patterns, that the fetus would have certain DQB1 or DRB1 alleles. If the non-maternal DQA1 sequence is, in fact, a sequencing error, then it is extremely unlikely that an independent sequencing error in the maternal DQB1 or DRB1 sequence would generate the expected DQB1 or DRB1 allele. This allows one to distinguish the fetal allele if the paternal alleles are unknown and distinguish this from a sequencing error in the maternal allele that gave rise to a sequence corresponding to a known HLA allele. For example, in a variation of this embodiment, sequences from genes DQB1 and DRB1 are analyzed simultaneously. In yet another embodiment, the invention comprises detecting a combination of HLA genes that includes two or more genes in linkage disequilibrium with each other and at least one gene not in linkage disequilibrium with the rest. For example, in a variation of this embodiment, sequences from genes DPB1, DQB1 and DRB1 are analyzed simultaneously. DPB1 is not in strong linkage disequilibrium with DQB1 or with DRB1 although DQB1 and DRB1 are in linkage disequilibrium with each other.

Simultaneous analysis can be performed in parallel reactions or by combining separate reactions in one, multiplex reaction, e.g., genomic PCR wherein several amplification primers are present in the same reaction volume. In some embodiments, the method of the invention comprises a sequencing step that enables quantitative detection of the maternal and non-maternal HLA alleles in the sample. In this embodiment, the method requires “deep sequencing” because only a small fraction (a few percent) of the total DNA in maternal plasma or blood is expected to be derived from the fetus. Next-generation sequencing (NGS) methods (also known as massive parallel sequencing (MPS) methods) clonally propagate in parallel millions of single DNA molecules. Each clonal population is then individually sequenced. Sometimes, NGS (MPS) methods are referred to as clonal sequencing. The advancement of the technology has allowed for ever longer sequence reads, up to 250 and more recently up to 700 nucleotides. However, cell-free fetal DNA is present in short fragments, the majority being about 160 base pairs long. (See U.S. application Ser. No. 12/940,992, filed on Nov. 5, 2010.) For such short target sequences, robust performance of the currently existing sequencing technology is assured.

The deep sequencing step of the method of the present invention requires a target enrichment step. In some embodiments, the target enrichment step comprises an amplification step. In other embodiments, other target enrichment methods are used, e.g. the library-based or probe-based methods of target enrichment described e.g., in U.S. Pat. No. 7,867,703 or 8,383,338. At least one round of amplification e.g., the first round may be performed by any method known in the art. In some embodiments, more than one round, e.g., two rounds of amplification are performed. In variations of this embodiment, subsequent rounds of amplification, e.g., amplification by PCR are performed using the same primers. In other variations of this embodiment, the primers differ by either extending further in the 3′-direction into the HLA sequence (nested primers) or by having additional sequences, e.g., non-HLA sequences, on the 5′-end.

In some embodiments, the enriched target is subjected to clonal amplification by any suitable method known in the art. In some embodiments, the clonal amplification comprises emulsion PR described in detail in the U.S. application Ser. No. 12/245,666, filed on Oct. 3, 2008, incorporated here by reference in its entirety for all purposes. Briefly, during emulsion PCR, the amplicons from the preceding rounds of amplification are contacted with a solid phase (e.g., beads) conjugated with an oligonucleotide capable of hybridizing to the amplicon, e.g., via hybridizing to the adaptor region of the primer used in a preceding round of amplification. As a result, the bead carries annealed amplicons hybridized to the adaptor region present on the bead. The beads are then suspended in an aqueous solution and oil is added to generate an emulsion. Each bead becomes suspended in an oil-enclosed microdroplet containing all the reagents necessary to carry out the clonal round of amplification. Each microdroplet encapsulates a reaction chamber for an amplification reaction. In variations of this embodiment, two types of beads are used: one type is conjugated to an oligonucleotide capable of hybridizing to one of the two strands of the amplicon; and the second type is conjugated to an oligonucleotide capable of hybridizing to the other strand of the amplicon. In other embodiments, the clonal amplification comprises a two-dimensional surface-based (e.g., slide-based) amplification as described e.g., in U.S. Pat. Nos. 7,835,871, 8,244,479, 8,315,817 and 8,412,467. In general, any method of clonal amplification that is available or will become available is within the scope of the invention.

The method of the invention comprises the use of primers targeting (i.e., specifically hybridizing to and capable of amplifying) portions of the sequences of HLA genes HLA-A, HLA-B, HLA-C, DRB1, DRB3, DRB4, DRB5, DQA1, DQB1, DPA1, and DPB1. In some embodiments, the primers target certain exons or introns of the HLA genes, e.g., HLA-A, exons 2 and 3; HLA-B, exons 2 and 3; HLA-C, exons 2 and 3; DQA1: exon2; DQB1: exons 2 and 3; DPA1: exon 2; DPB1: exon 2; DRB1: exon 2, DRB3: exon 2; DRB4: exon 2; and DRB5: exon 2. In other embodiments, the primers in a pair target a combination of exon and intron sequences. In some embodiments, one or more primers listed in Table I are used. As shown in Table 1, some primers are gene-specific, i.e., can amplify sequences from only one gene. Other primers are generic, i.e., can amplify sequences from more than one gene.

In some embodiments, to determine the fetal fraction in the sample, the amounts of maternal and non-maternal HLA alleles at the same locus are quantitatively determined by digital droplet PCR. Digital droplet PCR (ddPCR) enables absolute measure of a target nucleic acid in a sample, even at very low concentrations. The ddPCR method comprises the steps of digital dilution or droplet generation, PCR amplification, detection and (optionally) analysis. The droplet generation step comprises generation of a plurality of individual reaction volumes (droplets) each containing reagents necessary to perform nucleic acid amplification. The PCR amplification step comprises subjecting the droplets (or larger reaction volumes in which droplets have been deposited) to thermocycling conditions suitable for amplification of the nucleic acid targets to generate amplicons. The detection step comprises identification of droplets (or larger reaction volumes in which droplets have been deposited) that contain and do not contain amplicons. The analysis step comprises a quantitation that yields e.g., concentration, absolute amount or relative amount (as compared to another target) of the target nucleic acid in the sample.

The ddPCR step may be performed manually (i.e., with generic devices) or with a specialized device, such as e.g., ddPCR devices available from Bio-Rad Labs. (Hercules, Calif.), or RainDance Tech. (Billerica, Mass.) or similar devices that are or will become available. In some embodiments, the entire ddPCR step is performed with a specialized device. In other embodiments, one or more steps, e.g., digital dilution, thermocycling, detection and analysis are performed with a generic device selected from e.g., a manual or automated generic pipetting device, a thermocycler, an electrophoresis device and so on.

The detection of the ddPCR product may be performed by any generic or sequence-specific means of detecting nucleic acids. The detection may take place within the reaction volume or after an additional step, such as electrophoresis or chromatography. The detection may take place during amplification (real-time PCR) or after completion of amplification (endpoint PCR). A detectable label can be conjugated to a PCR reagent, such as a primer or probe. The label can be detectable by spectroscopic, photochemical, biochemical, immunochemical, chemical or other techniques can be used. To illustrate, useful labels include; radioisotopes, fluorescent dyes, electron-dense reagents, enzymes (as commonly used in ELISA), haptens, and proteins for which antisera or monoclonal antibodies are available. As an alternative to a labeled PCR reagent, the detection may be performed post-PCR with a separate labeled reagent, e.g., a sequence-specific labeled probe. Alternatively, a non-specific method of detecting nucleic acids, such as electrophoresis followed by staining can be used.

Unlike sequencing, the ddPCR based approach disclosed herein benefits from the knowledge of which HLA loci are informative (i.e., polymorphic) between the parents. In some embodiments, the method includes the first step of genotyping the parents to identify informative HLA loci. If such information is available, a single set of PCR reagents may be used to target the informative locus in the maternal plasma sample. Alternatively, without the paternal information, the maternal sample can be subjected to ddPCR analysis using one or more of the loci selected from HLA-A, HLA-B, HLA-C, DRB1, DRB3, DRB4, DRB5, DQA1, DQB1, DPA1, and DPB1 in the hope that at least one locus will be polymorphic between the mother and the fetus. In some embodiments, specific exons or portions of exons of HLA genes are targeted, e.g., HLA-A, exons 2 and 3; HLA-B, exons 2 and 3; HLA-C, exons 2 and 3; DQA1: exon2; DQB1: exons 2 and 3; DPA1: exon 2; DPB1: exon 2; DRB1: exon 2, DRB3: exon 2; DRB4: exon 2; and DRB5: exon 2. In other embodiments, the polymorphic HLA gene sequence comprises introns sequences or a combination of exon and intron sequences.

Currently used HLA primers aim to amplify and detect sequences of entire HLA exons. An average size of an HLA class II exon (exon 2) encoding the peptide binding groove is about 270 base pairs. Accordingly, a typical HLA typing assay involves amplicons of about 300 base pairs (Bentley, G., et al., (2009) High resolution, high throughput HLA genotyping by next-generation sequencing, Tissue Antigens, 74:393.) In contrast, due to the fragmented nature of the fetal DNA found in maternal blood or plasma, the primers in the present invention aim to amplify and detect the sequence of fragments no longer than 160 base pairs in length. The primers used in the present invention uniquely combine the ability to amplify the short cell-free DNA with the ability to target informative (i.e. most polymorphic) regions of the HLA genes. In some embodiments, the method of the present invention is practiced with primers including at least one primer having HLA-hybridizing regions listed in Table 1.

In some embodiments, the amplicons are sequenced by a base-incorporation method, e.g. a pyrosequencing method (U.S. Pat. Nos. 6,274,320, 6,258,568 and 6,210,891); a hydrogen ion detection method (ISFET) (e.g., U.S. Pat. No. 8,262,900), or a dye-terminator detection method (U.S. Pat. Nos. 7,835,871, 8,244,479, 8,315,817 and 8,412,467.) The HLA sequence data generated by the method of the present invention comprises HLA sequences of individual DNA molecules. A typical NGS instrument used in the method of the present invention (e.g., the GS family, 454 Life Sciences, Branford, Conn.; ION PROTON® and PGM™, Life Technologies, Grand Island, N.Y.; HISEQ® and MISEQ®, Ilumina, San Diego, Cal.) contains a data analysis module capable of quantitatively detecting each sequence present in the sample, e.g., each HLA allele sequence at the same locus present in the sample. The numbers of reads corresponding to fetal and maternal allele sequences are then counted to determine the fraction of the fetal allele and fetal DNA in the sample.

The computational step is typically performed by a computer capable of executing the functions of a software program. The present invention may be practiced with any suitable software that is available or will become available for analysis of individual nucleic acid sequence reads generated by clonal sequencing. The software may have specific features uniquely suitable for the analysis of HLA sequences and assignment of HLA genotypes. For example, software may compare the sequence reads obtained from a sample to a database of known HLA alleles. An example of such database is the IMGT/HLA sequence database maintained at the European Molecular Biology Laboratory (EMBL), see Robinson et al. (2003) IMGT/HLA and IMGT/MHC: sequence databases for the study of the major histocompatibility complex, Nucleic Acids Research, 31:311. The typical software for the analysis of HLA sequence reads identifies the majority groups among the reads present in the sample. In a typical human sample, no more than four groups of sequence reads are present for each HLA sequence tested: the forward and the reverse reads for each of the two HLA alleles at the same locus. (Only two sequences will be present in a sample is derived from a homozygous individual). In the context of the present invention, the software (as pre-programmed or with the input of the user through the user interface) performs an additional function of identifying a third allele: the non-maternal (fetal) HLA allele in addition to the two maternal HLA alleles at the same locus present in the sample. The software (as pre-programmed or with the input of the user through the user interface) must distinguish the minority non-maternal allele present at low concentration (typically between 1% and 15%) from the artifacts due to e.g., PCR misincorporations, sequencing errors, related pseudogenes, minor DNA contaminants in the sample, etc., that are present at a lower concentration than the non-maternal (fetal) allele, e.g. <<0.5%.

In some embodiments, the software compares the sequence reads to the HLA sequence database and identifies two (or one in case of a homozygous mother) most prevalent sequences as maternal alleles at a certain HLA locus. Not to limit the scope of the invention but merely by way of an example, the Conexio HLA genotyping software (Conexio Genomics, Ltd., Perth, Australia) compares the consolidated sequence reads that have been aligned with the reference sequence for a given amplicon (e.g. DQB1 exon 2) and compares the observed sequence reads with the IMGT/HLA sequence database. All the reads corresponding to that amplicon are sorted into the DQB1 exon 2 “bin” and the software assumes that there are only two allelic sequences (two forward and two reverse reads) for any one sample. These sequences are sorted into the “Master Layer” and used for the genotype assignment. All other sequence reads, typically much less abundant than the true alleles, corresponding to artifacts or contaminants, are sorted into the “Failed Layer” and are not used for genotype assignment.

The method of the present invention requires detection of more than one genotype, i.e. more than two alleles present in the sample. In some embodiments, the software (as pre-programmed or with the input of the user though a user interface) Identifies and sorts the minority components into multiple “bins” representing the different allelic groups corresponding to an amplicon, e.g., the DQB1 locus. For example, instead of having only one “bin” for DQB1 exon 2, the method comprises creation of multiple “bins” for multiple alleles at the DQB1 locus (e.g., DQB1*01:01, *02:01, *03:01, etc.). In some embodiments, more than 2, e.g., 3, 4, 5, 6 and as many as 15 or 20 of such bins are created. The sequences corresponding to the HLA type of the minority component are sorted into an appropriate bin. Noise (i.e. PCR and sequencing errors, pseudogenes, etc.) are still, in general, sorted into the Failed Layer for each bin. This step allows quick identification of the alleles of the majority component (maternal alleles), as well as identification of the reads corresponding to the minority component. Notably, this approach is suitable for identifying fetal alleles even if the parental genotype is not known. In some embodiments, the parental genotype is known. In such embodiments, the software may be modified to identify and count the specific maternal and paternal alleles and discard all reads that differ therefrom as “failed” reads.

In some embodiments, the method includes a step that minimizes errors resulting from artifacts due to e.g., PCR misincorporations, sequencing errors, related pseudogenes, minor DNA contaminants in the sample, etc. It is possible that a PCR or sequencing error could “convert” a maternal allele into another known HLA allele. Although this event is expected to be rare. i.e., at a frequency much lower than that of the non-maternal allele, in some embodiments, the method of the present invention includes a step of minimizing such errors. For example, the method may include analysis of two amplicons for genes that are in strong linkage disequilibrium, e.g., DQA1 and DQB1. If an error converted a majority allele into a sequence corresponding to a known third non-maternal allele for DQB1, it is extremely unlikely that a random error should also convert the maternal DQA1 allele into a sequence corresponding to the DQA1 allele in linkage disequilibrium with the artefactual DQB1 allele.

The present invention is a method of determining fraction or concentration of fetal nucleic acid in a sample comprising quantitatively detecting in the sample at least one non-maternal HLA allele, quantitatively detecting at least one maternal HLA allele at the same locus; and using the quantities of the maternal and non-maternal alleles, determining the fetal fraction in the sample. In some embodiments, at least one HLA allele is selected from HLA-A, HLA-B, HLA-C, DRB1, DRB3, DRB4, DRB5, DQA1, DQB1, DPA1, and DPB1 allele, specifically in some embodiments, the allele comprises sequences selected from HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2, or intron sequences or a combination of exon and intron sequences from these genes. In some embodiments, the quantitative detection comprises clonal sequencing. In some embodiments, the method comprises a target enrichment step prior to sequencing. In variations of this embodiment, the enrichment is performed by DNA amplification. In other variations of this embodiment, the enrichment is performed by target capture.

The method further comprises determining the fetal fraction in the sample using the quantities of maternal and non-maternal sequence reads. The determining step comprises calculating the ratio of the non-maternal (fetal) reads to the maternal reads or to the total number of reads minus background noise. Because the ratio reflects the proportion of only one of the two fetal alleles, the ratio is doubled to obtain the fraction of fetal DNA.

Without limiting the invention to a single technology or instrument but merely by way of example, an embodiment of the method may be performed using the GS family of sequencing instruments including GS FLX®, GS FLX+®, GS FLX TITANIUM® or GS Junior® (454 Life Sciences, Branford, Conn.) as described below.

In this embodiment, the target enrichment and sequencing steps comprise: (a) in the first amplification reaction, amplifying the exons or introns of one or more HLA-A, HLA-B, HLA-C, DRB1, DRB3, DRB4, DRB5, DQA1, DQB1, DPA1, and DPB1 genes that comprise polymorphic sites using amplification primers comprising the following sequences listed in the 5′- to 3′-prime direction: an adapter sequence, a molecular identification sequence, and an HLA-hybridizing sequence; (b) in the second amplification reaction, performing emulsion PCR; (c) determining the sequence of each amplicon obtained in step (b) using pyrosequencing; (d) assigning the HLA alleles to the mother or the fetus by comparing the sequence of the HLA amplicons determined in step (c) to the known HLA sequences to determine which HLA alleles are present in the maternal blood or plasma; (e) for one or more HLA alleles, quantifying the number of fetal and maternal sequencing reads corresponding to each allele obtained in step (c); and (f) using the quantity obtained in step (e), determining the fraction of the fetal DNA present in the maternal blood or plasma by calculating a ratio of the non-maternal (fetal) reads to the maternal reads or to the total number of reads minus the background or to the total number of reads.

In variations of this embodiment, the method comprises after step (a), pooling amplicons from multiple individuals and performing the subsequent steps (b)-(c) on a pool of amplicons from multiple individuals. In this variation, the amplification primer further comprises an individual identification tag also known as multiplex identification (MID) tag.

In other embodiments, steps (b)-(e) or equivalents thereof are performed using any available deep sequencing technology and instrument (i.e., technology and instrument capable of digital sequence readout). Without limitation, the examples of instruments include GS family of instruments (454 Life Sciences, Branford, Conn.); ION PROTON® and PGM™ (Life Technologies, Grand Island, N.Y.); HISEQ® and MISEQ® (Illumina, San Diego, Calif.) or any improvements and modifications of thereof.

In some embodiments, determination of the fetal fraction comprises comparison of the reads corresponding to the non-maternal (fetal) allele to the sum of all reads at the same locus or to the reads corresponding to the maternal allele at the same locus obtained from the sample or to the sum of the maternal alleles plus the fetal alleles. In some embodiments, the comparison step comprises calculating 2× the ratio of the reads corresponding to the non-maternal (fetal) allele to the sum of all reads at the same locus obtained from the sample; or to the reads corresponding to the maternal allele at the same locus obtained from the sample or to the sum of the maternal alleles plus the fetal alleles. For example, in one embodiment, if the numbers of reads for the maternal alleles are M1 and M2 and the non-maternal fetal allele is F, the fetal fraction (FF) could be determined according to Formula 1:

FF=2×F/(M1+M2+F) Formula 1

In another embodiment, the reads can be broken down into the forward (F) and reverse (R) sequencing reads for each allele. Then the fetal fraction (FF) could be determined as average of reverse (FF_{R}) and forward (FF_{F}) fractions determined according to Formula 2:

FF_{R}=2×F_{R}/(M1_{R}+M2_{R}+F_{R})

FF_{F}=2×F_{F}/(M1_{F}+M2_{F}+F_{F})

FF=(FF_{R}+FF_{F})/2 Formula 2

Any number of similar formulas that determine fetal fraction using the non-maternal fetal allele F and one or both of maternal alleles M1 and M2 or a single maternal allele M in the case of a homozygous mother, can be devised and are within the scope of the invention.

In yet another embodiment, several HLA loci can be sequenced. The reads from each locus can be used to calculate fetal fraction according to Formula 1 or Formula 2 and the resulting fetal fraction values for each locus can be averaged to obtain an estimate of the fetal fraction.

Within the scope of the present invention are also various diagnostic and monitoring methods that require determination of the fetal fraction or amount of fetal DNA in maternal blood or plasma. In one embodiment, the invention is a method of determining whether a pregnant patient has or is likely to develop preeclampsia. The method comprises determining whether the fetal fraction (concentration of fetal nucleic acid in the patient's blood) exceeds a threshold level. In this embodiment, the concentration of fetal nucleic acid is determined by a method comprising obtaining a blood sample from the patient; quantitatively detecting in the sample at least one non-maternal HLA allele in the sample; optionally, quantitatively detecting in the sample at least one maternal HLA allele al the same locus; and optionally comparing the quantities of the maternal and non-maternal HLA alleles thereby determining fraction or concentration of fetal nucleic acid in the sample. If the fetal fraction is found to exceed a certain predetermined level, the patient is diagnosed as having or likely to develop preeclampsia. The predetermined level can be for example, fetal fraction or amount of fetal DNA in maternal blood or plasma of a patient without preeclampsia in the same gestational stage.

In variations of this embodiment, the invention comprises a method of monitoring a pregnant patient for development of preeclampsia by periodically determining the fetal fraction or concentration of fetal nucleic acid in the patient's blood or plasma determined by a method comprising obtaining a blood sample from the patient; quantitatively detecting in the sample at least one non-maternal HLA allele in the sample; optionally, quantitatively detecting in the sample at least one maternal HLA allele at the same locus; optionally comparing the quantities of the maternal and non-maternal HLA alleles thereby determining the fetal fraction or concentration of fetal nucleic acid in the sample. If periodic measurement detects an increase, the patient is diagnosed as having or likely to develop preeclampsia.

In other embodiments, the invention is a method of detecting a fetal chromosomal abnormality. The method comprises determining fetal fraction (concentration of fetal DNA in maternal blood) by obtaining a blood sample from the mother, quantitatively detecting at least one non-maternal HLA allele in the sample; quantitatively detecting in the sample at least one maternal HLA allele at the same locus; comparing the quantities of the maternal and non-maternal HLA alleles thereby determining fraction of fetal nucleic acid in the sample. The method further comprises quantitatively detecting in the sample a locus from at least one chromosome suspected of an abnormality; determining whether the detected chromosomal locus is present in an abnormal amount relative to the fraction of fetal DNA determined in step thereby detecting the fetal chromosomal abnormality. The abnormal amount is defined as the amount substantially different from the amount of the same locus found in maternal blood of euploid fetuses at the same gestational stage.

In another embodiment, the invention is a method of detecting a presence or a homozygous state of an allele in a fetus wherein the allele is associated with a disease state. The disease state comprises an existing disease or a predisposition to developing the disease. In one variation of this embodiment, the method comprises detecting the presence of a single allele representing a carrier state of the fetus carrying one recessive allele. In another variation of this embodiment, the method comprises detecting the presence of a single allele representing a disease state of the fetus carrying an autosomal dominant allele or a haploinsufficient allele. In another variation of this embodiment, the method comprises detecting in the fetus a homozygous state of a recessive allele that is associated with a disease state of the fetus.

The method comprises obtaining a blood sample from the mother carrying the fetus; determining a fraction of a non-maternal HLA allele in the sample by a method comprising quantitatively detecting at least one non-maternal HLA allele in the sample; quantitatively detecting in the sample at least one maternal HLA allele at the same locus; comparing the quantities of the maternal and non-maternal HLA alleles thereby determining the fraction of the non-maternal HLA allele in the sample; quantitatively detecting in the sample the allele associated with the disease state; comparing the quantity of the allele associated with the disease state with the fraction of the non-maternal HLA allele thereby determining whether said allele associated with disease state is present in a single copy, two copies or is absent from the fetus.

### EXAMPLES

### Example 1

### Sample Collection and DNA Purification

Samples were collected from human subjects: a woman in the third trimester of pregnancy and the father of the fetus. The DNA was prepared as follows. Whole blood was collected and processed within two weeks. “Buffy coat” was prepared by centrifugation at ambient temperature at 1600×g, 10 min. Plasma (supernatant) was removed carefully to avoid any cells and re-centrifuged at 16,000×g for 10 min. The cell free plasma was carefully removed without disturbing the cell pellet and stored at −80° C. DNA was prepared from the “buffy coat” by use of the QIAGEN QIAMP® DNA Blood Mini Kit (Qiagen, Valencia, Calif.), and from the cell-free plasma as per the COBAS® EGFR Mutation Test Kit: EDTA Plasma protocol (Roche Applied Science, Indianapolis, Ind.) per manufacturers' instructions. Saliva was collected using the Oragene-Dx kit (DNA Genotek, Kanata, Ont.) and DNA was isolated according to per manufacturer's instructions. DNA was purified from cell lines using the Gentra PUREGENE® kit (Qiagen, Valencia, Calif.).

### Example 2

### Genotyping of Parents

Genotyping of parents was performed using DNA from buffy coat or saliva isolated in Example 1, either by the method published by Moonsamy, P., et al. (2013) Tissue Antigens, 81:141, or as described for the GS GType HLA primer HR kit. DQB1 locus was found to be informative: the father possessed a DQB1 allele absent from the mother. Parental genotypes were as follows:
Maternal HLA Genotype: DQB1*03:01, *06:03
Paternal HLA Genotype: DQB1*06:02, *06:02

### Example 3

### Deep Sequencing of Cell-Free DNA from Plasma

PCR amplifications were carried out in individual 25 μl reactions with 1-10 ng of DNA template and 10 pmoles each of forward and reverse primer, 10 mM Tris-HCl, pH 8.3, 50 mM KCl, 1.5 mM MgCl, 150 μM each of dA, dC, dG and dUTP, glycerol 10% w/v, and AmpliTaq Gold® DNA polymerase. Thermal cycling conditions were: 95° C.-10 min; 31 cycles of 95° C.-15 sec., 60° C.-45 sec, 72° C.-15 sec”; 72° C.-5 min. in an ABI GeneAmp® PCR System 9700.

The primers for use with the GS FLX® instrument had the following arrangement in the 5′-3′-orientation: Adaptor-Key tag-MID-HLA-hybridizing sequence. The primers for use with the MI-SEQ® instrument had the following arrangement in the 5′-3′-orientation: Adaptor-MID-HLA-hybridizing sequence. The adaptor, key tag and MID sequences were designed according to the manufacturers' recommendations. The HLA-hybridizing sequences are listed in Table 1.

TABLE 1
HLA-hybridizing sequences of the primers

| | | |
|---|---|---|
| | SEQ ID | |
| Locus/Exon | NO: | Sequence |

Forward Primers

| | | |
|---|---|---|
| DPB1/Exon 2 | SEQ ID | GCTGCAGGAGAGTGGCGCCTCCGCTCAT |
| | NO: 1 | |
| DPB1/Exon 2 | SEQ ID | ACGGAGCTGGGGCGGCC |
| | NO: 2 | |
| DPB1/Exon 2 | SEQ ID | GAGAGTGGCGCCTCCGCTCAT |
| | NO: 3 | |
| DPB1, DRB1, | SEQ ID | ACGGAGCTGGGGCGGCC |
| DRB3/4/5; | NO: 4 | |
| Exon 2 | | |
| DQB1 Exon 2 | SEQ ID | AGGATCCCCGCAGAGGATTTCGTGTACCA |
| | NO: 5 | |
| DQB1 Exon 2 | SEQ ID | TCCCCGCAGAGGATTTCGTGTACCA |
| | NO: 6 | |

Reverse Primers

| | | |
|---|---|---|
| DPB1/Exon 2 | SEQ ID | CGGATCCGGCCCAAAGCCCTCACTC |
| | NO: 7 | |
| DPB1/Exon 2 | SEQ ID | CCGGCCCAAAGCCCTCACTC |
| | NO: 8 | |
| DPB1/Exon 2 | SEQ ID | CCGGCCCAAAGCCCTCACTC |
| | NO: 9 | |
| DPB1, DRB1, | SEQ ID | AGCTCCGTCACCGCCCGGAA |
| DRB3/4/5; | NO: 10 | |
| Exon 2 | | |
| DQB1 Exon 2 | SEQ ID | CGGTACACCTCCACGTCGCTGTCSAA |
| | NO: 11 | |
| DQB1 Exon 2 | SEQ ID | CGGTACACCTCCACGTCGCTGTCSAA |
| | NO: 12 | |

*S = C or G.
The primer sample contains a mixture of equal amounts of oligonucleotides containing C or G at the indicated position.

Amplicon cleanup, quantification, dilution and pooling were performed as follows. Short non-specific and primer-dimer artifact products were removed from the amplicons using the AMPURE® system (Agencourt Bioscience Corp., Beverly, Mass.), following the protocol for cleanup described in the 454 Life Sciences GS GType HLA MR and HR kits (Roche Applied Science, Indianapolis, Ind.). Aliquots of purified amplicons were further evaluated by electrophoresis on a 96 well E-GEL® (Life Technologies, Carlsbad, Calif.). If primer dimers were observed the AMPure step was repeated and product reevaluated by E-GEL®. The purified amplicons were then quantified by QUANT-IT™ PICOGREEN® assay (Life Technologies, Carlsbad, Calif.) on a microplate spectrofluorimeter. Eight standards spanned DNA concentrations from 0 ng/μl to 12.5 ng/μl. Any amplicons that could not be detected by PICOGREEN® were assigned a concentration of 0.1 ng/μl (in order to allow a dilution calculation to be made) and carried through subsequent steps. Amplicons were diluted to 1×10⁶ molecules/μl. Pools of amplicons were made such that all amplicons destined for sequencing on a single region of the 454 PicoTiter Plate (PTP) were pooled; in general 5 ul of each amplicon was added to a pool.

Emulsion PCR, bead recovery and pyrosequencing were performed as follows. Emulsion PCR (emPCR), enrichment of DNA containing beads, and pyrosequencing on the GS FLX® instrument (454 Life Sciences, Branford, Conn.) were carried out on a 4-region PTP as per the GS FLX TITANIUM® Series manuals: emPCR Method Manual—LibA MV (January 2010); Sequencing Method Manual (May 2010), with the following exceptions: 1) during emPCR, amplicon pools were used at 0.4-0.5 copies per bead, 2) the emPCR primer was used at a concentration of 0.25 times that specified, 3) bead enrichment was automated by use of the REMe module (Roche Applied Science, Indianapolis, Ind.) on a MultiProbe HT liquid handler (Perkin Elmer, Waltham, Mass.), and 4) for sequencing, 60% of the recommended load of enriched DNA beads was dispensed onto the PTP plate. Sequencing on the GS JUNIOR® instrument was carried out in the same way except that the method manuals used were GS JUNIOR® cmPCR Amplification Method Manual Lib-A_March 2012 and GS Junior Sequencing Manual_Jan2013.

Sequences were consolidated using the consensus functions of 454 AVA® software. ASSIGN ATF® 454 software (v 34) (Conexio Genomics, Ltd., Perth, Australia) installed on a Microsoft Windows' based computer, was used for analysis of sequences. The software assigned the alleles to each of the sequence reads and computed the number of sequence reads corresponding to each allele. Results are shown in Table 2. The column “valid reads” shows the number for reads where a sequence was successfully read and identified as one of the parental alleles of the HLA DQB1 gene.

TABLE 2
Sequence reads obtained in Example 3

| Locus | Amplicon | Direction | Valid reads |
|---|---|---|---|
| DQB1*3:01 | Exon 2 | F | 13651 |
| | Exon 2 | R | 9394 |
| DQB*6:02 | Exon 2 | F | 1506 |
| | Exon 2 | R | 1032 |
| DQB1*6:03 | Exon 2 | F | 10351 |
| | Exon 2 | R | 7785 |

Fetal fraction was calculated as double the ratio of the reads corresponding to the non-maternal (fetal) allele to the sum of all reads obtained from the sample as shown in Table 3.

TABLE 3
Determination of fetal fraction

| | | | % of Total |
|---|---|---|---|
| Allele | Derivation | Reads | Reads |
| DQB1*03:01 | Maternal | 23045 | 52.7 |
| DQB1*06:03 | Maternal | 18136 | 41.5 |
| DQB1*06:02 | Fetal | 2538 | 5.8 |

The calculation determined that 5.8% of the HLA DQB1 sequences obtained were from the paternal allele, representing a haploid genome equivalent. Accounting for diploid fetal genome, 5.8×2=11.6% of the plasma DNA was fetal.

### Example 4

### Detecting the Presence or a Homozygous State of a Allele

In this prophetic example, a patient is carrying a fetus where both parents are carriers of the same mutant allele of the Cystic Fibrosis Transmembrane Regulator (CFTR) gene (genotype Dd). The method of the present invention enables determination whether the fetus is mutation-free, is a carrier or is homozygous for the mutation and will be affected with cystic fibrosis (CF). The first step comprises determination of the fetal fraction in a maternal blood sample by a method described in Examples 1 and 3, wherein maternal HLA alleles are H1 and H2 and the non-maternal HLA allele at the same locus is H3. Based on the date in Table 4, the fetal fraction in the sample is 2×2=4%.

TABLE 4
Hypothetical fraction of the fetal HLA allele in the sample

| | | % of Total |
|---|---|---|
| Allele | Derivation | Reads |
| H1 | Maternal | 50 |
| H2 | Maternal | 48 |
| H3 | Fetal | 2 |

The second step comprises determining the fractions of mutant CFTR alleles (d) and non-mutant CFTR alleles (D) in the same sample by any sequencing method, e.g., the method described in Example 3. The following is a hypothetical determination of the CF status based on the fetal fraction determined to be 4% in the sample (hypothetical data in Table 4). According to the hypothetical data presented in Table 5, if the mutant (d) allele is present at 52% and the non-mutant (D) allele is present at 48%. The excess of the mutant allele in the sample is 4% which corresponds to the fetal fraction determined in the same sample. Therefore the fetus likely carries only the mutant alleles (genotype dd) and is affected with cystic fibrosis.

TABLE 5
Hypothetical fractions of the CFTR alleles in the sample

| | | % of | % of Total |
|---|---|---|---|
| Allele | Derivation | Alleles | Reads |
| D | Maternal | 48 | D - 48 |
| d | Maternal | 48 | d - 52 |
| d | Fetal | 2 | |
| d | Fetal | 2 | |

### Example 5

### Cell Line Control Samples

In the absence of true clinical controls for ddPCR, DNA from cell lines identified to be matched to each of the four known parental alleles under the probe binding region of the HLA-DQB1 were used (Table 6).

TABLE 6
Cell lines with sequence in the probe binding
region identical to the indicated alleles

| Allele | Representative cell line |
|---|---|
| M1 | AMALA |
| M2 | ARENT |
| P | AMAL |

The cell line DNA was diluted and/or blended to create contrived pure maternal or paternal DNA controls. Similarly, DNA samples from cell lines matched to the maternal alleles (M1 and M2) were spiked with those matched to paternal alleles to create 10% and 2.5% paternal DNA blends. All the samples were characterized using a two TaqMan minor groove binder (MGB) probe assay that is specific to one maternal allele and the single paternal allele (homozygous father). The FAM-labeled probe is perfectly matched to the M1 allele with 2-3 mismatches against M2 and P alleles. The Vic-labeled probe is perfectly matched to the paternal allele with 1-2 mismatches against each of the maternal alleles. Primers for a short amplicon of 139 base pairs were designed in a conserved region well matched to all alleles (Table 7.)

TABLE 7
Oligonucleotide sequences

| Descrip- | SEQ ID | | |
|---|---|---|---|
| tion | NO: | Sequence | Label |
| Forward | SEQ ID | TCCCCGCAGAGGATTTCGTGTA | |
| primer | NO: 13 | CCA | |
| Reverse | SEQ ID | CGGTACACCTCCACGTCGCTGT | |
| primer | NO: 14 | CSAA (S = C or G) | |
| M1 matched | SEQ ID | CGTGCGTTATGTGACC | FAM-MGB |
| probe | NO: 15 | | |
| P matched | SEQ ID | CGTGCGTCTTGTGAC | VIC-MGB |
| probe | NO: 16 | | |

### Example 6

### Digital Droplet PCR (ddPCR)

The ddPCR setup was done per manufacturer's instructions for QX100™ Droplet Generator (BioRad Labs., Hercules, Calif.). Each sample was run in duplicate reactions. 9 uL of sample was combined with BioRad Droplet PCR Supermix, 250 nM of each probe, and 900 nM of each primer, and 4 units of uracil-N-glycosylase (UNG) in a final 20 μL PCR volume. The final reaction mixture was transferred into a single well on a droplet generator chip along with 70 uL of droplet generator oil in parallel wells. Upon completion of droplet generation, 40 uL of the resulting droplets suspended in oil was transferred to a 96 well plate. Droplets were then cycled using the following thermal cycling profile: 50° C. for 5-minutes (UNG step), followed by a 10-minute heat activation step at 95° C., and 40 cycles of 94° C. (30-seconds) to 57° C. (1-minute), and a 10-minute 98° C. hold. Endpoint fluorescence for each droplet was read in both the FAM and VIC channels using the BioRad QX100™ droplet reader. In order to avoid false positives, thresholds for positive droplet calls were drawn above any noise observed in no target control replicates and/or pure maternal cell line controls (in the VIC channel) and pure paternal cell line controls (in the FAM channel). The copies/μL concentration output from merged wells of two replicates per sample was converted to droplets per reaction by multiplying by 20.

Percentage of fetal DNA (fetal fraction) was determined by dividing the VIC positives (detects the paternal allele) by the total signal (VIC positives+2×FAM M1 positives). Detection of any paternal alleles in a maternally derived sample is indicative of circulating fetal DNA in the maternal bloodstream. Since only one paternal allele is passed onto the fetus, the VIC signal must be doubled to obtain the correct fetal fraction. The results are shown in Table 8. The FAM channel represents maternal allele detection and the VIC channel represents paternal allele detection in samples 1-6.

The data in Table 4 shows the ability of this ddPCR assay to detect paternal alleles at 2.5 and 10% in a background of maternal DNA; as well as determination of the fetal fraction in a clinical plasma sample from a mother in the third trimester of pregnancy was calculated as double the fraction of the paternal allele.

TABLE 8
Determined maternal and paternal allele fraction and or fetal fraction

| | | Observed M1 | Observed P | | % paternal | |
|---|---|---|---|---|---|---|
| Sample | | allele | allele | % maternal DNA* | allele | % fetal DNA |
| No. | Description | (copies/rxn) | (copies/rxn) | (M1 × 2;)/(total**) | P/(total) | % P × 2 |
| 1 | No Target Control | 0 | 0 | 0 | 0 | N/A |
| 2 | Paternal control | 0 | 2000 | 0 | 100 | N/A |
| | 2000 c P cell line DNA | | | | | |
| 3 | Maternal control: | 1016 | 0 | 100 | 0 | N/A |
| | 1000 c M1 + 1000 c M2 cell | | | | | |
| | line DNA blend | | | | | |
| 4 | 10% P control: | 972 | 192.8 | 91.0 | 9.0 | N/A |
| | 200 c of P and 1000 c each | | | | | |
| | of M1, M2 cell line DNA | | | | | |
| | blend | | | | | |
| 5 | 2.5% P control: | 1000 | 49 | 97.8 | 2.2 | N/A |
| | 50 c of P and 1000 c each | | | | | |
| | of M1, M2 cell line DNA | | | | | |
| | blend | | | | | |
| 6 | Clinical Sample: 3* | 48.6 | 6.52 | 88.2 | 5.91 | 11.82 |
| | trimester maternal plasma | | | | | |

*% maternal is double of the % of a single allele (M1 × 2) in a heterozygous mother (genotype M1 M2)
**“Total” for contrived samples is (2 × M1 + P). It would have been (2 × M1 + 2 × P1) if the father was heterozygous (genotype P1 P2).
“Total” for the clinical sample is (2 × M1) + (2 × P) to account for the diploid mother and a diploid fetus where the M1 allele was not transmitted to the fetus. It would have been 2 (M1 − P) + (2 × P) where the M1 allele was transmitted to the fetus. In this example, genotyping the baby confirmed that M1 was not transmitted to the fetus.

While the invention has been described in detail with reference to specific examples, it will be apparent to one skilled in the art that various modifications can be made within the scope of this invention. Thus the scope of the invention should not be limited by the examples described herein, but by the claims presented below.

We claim:

1. A method of determining a fraction of fetal nucleic acid in a blood or plasma sample comprising:
(a) quantitatively detecting at least one non-maternal HLA allele in the sample;
(b) quantitatively detecting in the sample at least one maternal HLA allele at the same locus;
(c) using the quantities detected in step (a) and (b), determining fraction of fetal nucleic acid in the sample.

2. The method of claim 1, wherein the determining step comprises calculating 2 times the ratio of the quantity of the non-maternal HLA allele detected in step (a) to the sum of quantities of HLA alleles determined in steps (a) and (b).

3. The method of claim 1, wherein the at least one non-maternal and maternal HLA allele is selected from HLA-A, HLA-B, HLA-C, DRB1, DRB3, DRB4, DRB5, DQA1, DQB1, DPA1, and DPB1 allele.

4. The method of claim 1, wherein the at least one non-maternal and maternal HLA allele comprises sequences selected from HLA-A, exons 2 and 3; HLA-B, exons 2 and 3; HLA-C, exons 2 and 3; DQA1, exon 2; DQB1, exons 2 and 3; DPA1, exon 2; DPB1, exon 2; DRB1, exon 2; DRB3, exon 2; DRB4, exon 2; and DRB5, exon 2 or an intron sequence from said HLA genes or a combination of exon and intron sequences from said genes.

5. The method of claim 1, wherein said HLA alleles are quantitatively detected by a method comprising clonal sequencing.

6. The method of claim 1 further comprising a target enrichment step prior to sequencing.

7. The method of claim 6, wherein the target enrichment step comprises at least one round of genomic DNA amplification.

8. The method of claim 6, wherein enrichment comprises target capture.

9. The method of claim 5, wherein clonal sequencing includes a step of clonal amplification.

10. The method of claim 9, wherein said clonal amplification step is performed with a forward primer and reverse primer, each primer comprising an adapter sequence and an HLA-hybridizing sequence.

11. The method of claim 1, wherein said HLA alleles are quantitatively detected by a method comprising:
(a) amplification with a forward primer and reverse primer to obtain HLA amplicons;
(b) performing clonal sequencing to determine the sequence of the HLA amplicons obtained in step (a);
(c) identifying at least one maternal HLA allele and at least one non-maternal HLA allele at the same locus;
(d) comparing the number of maternal and non-maternal HLA sequence clones identified in step (c) thereby determining fraction of fetal nucleic acid in the sample.

12. The method of claim 11, wherein identifying in step (c) comprises computational steps of:
(a) comparing the sequences at the HLA locus to an HLA sequence database;
(b) sorting the sequences into multiple bins corresponding to known HLA alleles;
(c) identifying one or two majority sequences as maternal alleles;
(d) identifying one or two most represented minority sequences as non-maternal alleles.

13. The method of claim 11, wherein determining the sequence of the HLA amplicons in step (b) comprises sequencing by synthesis.

14. The method of claim 11, wherein the comparing step comprises calculating a double of a ratio of the non-maternal HLA allele to the total number of HLA alleles at the same locus identified in step (d).

15. The method of claim 11, wherein in step (a) the non-maternal HLA allele and the maternal HLA allele at the same locus are detected at two, three or more loci.

16. The method of claim 15, wherein said three or more loci comprise sequences from genes DPB1, DQB1 and DRB1.

17. The method of claim 15, wherein the non-maternal and maternal HLA alleles at two, three or more loci are simultaneously amplified in the same reaction volume by multiplex PCR.

18. The method of claim 1, wherein the HLA alleles are quantitatively detected by a method comprising:
(a) partitioning the sample into a plurality of reaction volumes, each comprising between zero and approximately five copies of the target HLA allele;
(b) assaying each reaction volume for the presence of the target HLA allele;
(c) comparing the number of reaction volumes containing the non-maternal HLA allele to the number of reaction volumes containing the maternal HLA allele at the same locus, thereby determining fraction of the fetal nucleic acid in the sample.

19. The method of claim 18, wherein assaying in step (b) comprises amplification by digital PCR.

20. The method of claim 1, further comprising independently obtaining genotype information for one or both parents at the at least one HLA locus.

21. A method of detecting a chromosomal abnormality in a fetus comprising:
(a) obtaining a blood sample from the mother carrying the fetus;
(b) determining fraction of fetal nucleic acid in the sample by a method comprising quantitatively detecting at least one non-maternal HLA allele in the sample; quantitatively detecting in the sample at least one maternal HLA allele at the same locus; comparing the quantities of the maternal and non-maternal HLA alleles thereby determining concentration of fetal nucleic acid in the sample;
(c) quantitatively detecting in the sample a locus from at least one chromosome suspected of an abnormality;
(d) determining whether the chromosomal locus detected in step (c) is present in an abnormal amount relative to the concentration of fetal DNA determined in step (b) thereby detecting the chromosomal abnormality.

22. The method of claim 21, wherein the at least one non-maternal HLA allele is selected from HLA-A, HLA-B, HLA-C, DRB1, DRB3, DRB4, DRB5, DQA1, DQB1, DPA1, and DPB1 allele.

23. The method of claim 21, wherein the at least one non-maternal HLA allele is selected from HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2 or a combination of exon and intron sequences from said genes.

24. The method of claim 21, wherein in step (a) the non-maternal HLA allele and the maternal HLA allele at the same locus are detected at two, three or more loci.

25. The method of claim 24, wherein said three or more loci comprise sequences from genes DPB1, DQB1 and DRB1.

26. A method of determining whether a pregnant patient has or is likely to develop preeclampsia by determining whether fetal fraction in the patient's blood exceeds a threshold level, wherein the fetal fraction is determined by a method comprising obtaining a blood sample from the patient; quantitatively detecting at least one non-maternal HLA allele in the sample; quantitatively detecting in the sample at least one maternal HLA allele at the same locus; comparing the quantities of the maternal and non-maternal HLA alleles thereby determining fetal fraction in the sample.

27. The method of claim 26, wherein the at least one non-maternal HLA allele is selected from HLA-A, HLA-B, HLA-C, DRB1, DRB3, DRB4, DRB5, DQA1, DQB1, DPA1, and DPB1 allele.

28. The method of claim 26, wherein the at least one non-maternal HLA allele is selected from HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2 or a combination of exon and intron sequences from said genes.

29. The method of claim 26, wherein in step (a) the non-maternal HLA allele and the maternal HLA allele at the same locus are detected at two, three or more loci.

30. The method of claim 29, wherein said three or more loci comprise sequences from genes DPB1, DQB1 and DRB1.

31. A method of monitoring a pregnant patient for development of preeclampsia by periodically determining fetal fraction in the patient's blood by the method of claim 26 and if an increase in the fetal fraction is detected, diagnosing the patient as having or likely to develop preeclampsia.

32. A method of determining whether a pregnant patient has or is likely to develop preeclampsia by determining whether concentration of fetal nucleic acid in maternal blood exceeds a threshold level, wherein the concentration of fetal nucleic acid is determined by a method comprising obtaining a volume blood sample from the patient; quantitatively detecting at least one non-maternal HLA allele in the volume of the sample thereby determining the concentration of fetal nucleic acid.

33. The method of claim 32, wherein the at least one non-maternal HLA allele is selected from HLA-A, HLA-B, HLA-C, DRB1, DRB3, DRB4, DRB5, DQA1, DQB1, DPA1, and DPB1 allele.

34. The method of claim 32, wherein the at least one non-maternal HLA allele is selected from HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2 or a combination of exon and intron sequences from said genes.

35. The method of claim 32, wherein in step (a) the non-maternal HLA allele and the maternal HLA allele at the same locus are detected at two, three or more loci.

36. The method of claim 35, wherein said three or more loci comprise sequences from genes DPB1, DQB1 and DRB1.

37. A method of detecting a presence or a homozygous state of an allele in a fetus wherein the allele is associated with a disease state, the method comprising:
(a) obtaining a blood sample from the mother carrying the fetus;
(b) determining a fraction of a non-maternal HLA allele in the sample by a method comprising quantitatively detecting at least one non-maternal HLA allele in the sample; quantitatively detecting in the sample at least one maternal HLA allele at the same locus; comparing the quantities of the maternal and non-maternal HLA alleles thereby determining the fraction of the non-maternal HLA allele in the sample;
(c) quantitatively detecting in the sample the allele associated with the disease state;
(d) comparing the quantity of the allele associated with the disease state with the fraction of the non-maternal HLA allele thereby determining whether said allele associated with disease state is present in a single copy, two copies or is absent from the fetus.

38. The method of claim 37, wherein the at least one non-maternal HLA allele is selected from HLA-A, HLA-B, HLA-C, DRB1, DRB3, DRB4, DRB5, DQA1, DQB1, DPA1, and DPB1 allele.

39. The method of claim 37, wherein the at least one non-maternal HLA allele is selected from HLA-A, exons 2 and 3; HLA-B, exons 2 and 3; HLA-C, exons 2 and 3; DQA1, exon2; DQB1, exons 2 and 3; DPA1, exon 2; DPB1, exon 2; DRB1, exon 2; DRB3, exon 2; DRB4, exon 2; and DRB5, exon 2 or a combination of exon and intron sequences from said genes.

40. The method of claim 37, wherein in step (a) the non-maternal HLA allele and the maternal HLA allele at the same locus are detected at two, three or more loci.

41. The method of claim 40, wherein said three or more loci comprise sequences from genes DPB1, DQB1 and DRB1.
